(19) United States
(12) Patent Application Publication
(10) Pub. No.: US 2018/0374562 A1
(43) Pub. Date: Dec. 27, 2018

(54) METHOD AND APPARATUS FOR FACILITATING NETWORK ACCESS SHARING BY PATIENT GATEWAYS

(71) Applicant: Nokia Technologies Oy, Espoo (FI)

(72) Inventor: Teemu Ilmari SAVOLAINEN, Nokia (FI)

(21) Appl. No.: 16/063,661

(22) PCT Filed: Dec. 23, 2015
(86) PCT No.: PCT/IB2015/059961

§ 371 Date: Jun. 18, 2018

(51) Int. Cl. G16H 10/60; H04W 40/12; H04W 24/08; H04W 4/38

(52) U.S. Cl. CPC G16H 10/60; H04W 4/38; H04W 24/08; H04W 40/12

(57) ABSTRACT

A method, apparatus and computer program product are provided in accordance with an example embodiment in order to provide communication of patient sensor data from patient gateways to a network device and, more particularly, to providing for such communication in an energy efficient manner. In the context of a method, the method includes collecting patient sensor data at a first patient gateway, monitoring for a second patient gateway and determining one or more characteristics of each second patient gateway. The method also includes analyzing the one or more characteristics to determine which of the first or second patient gateways should serve as a cellular uplink gateway. In an instance in which a respective second patient gateway is selected, the method causes a representation of the patient sensor data to be provided to the respective second patient gateway for subsequent provision to the network device.

### TECHNOLOGICAL FIELD

Example embodiments relate generally to the communication of patient sensor data from patient gateways to a network device and, more particularly, to providing for such communication in an energy efficient manner.

### BACKGROUND

In today's world, some hospital patients wear or are otherwise associated with patient gateways. These patient gateways may include or otherwise be in communication with one or more sensors. The patient gateways may also include uplink cellular radio for communicating with a cloud server or other remote network, and one or more local connectivity radios, such as may operate in accordance with Bluetooth™ Low Energy (LE), Bluetooth™, or WiFi protocols. The sensors are used for monitoring a patient and, in some instances, the patient may be wearing additional sensors that are connected to gateway via wires or wireless connections. Data from these gateways is transmitted to and collected into a cloud server for further analysis and use. In this regard, the sensors included within or connected to the patient gateways may produce sensory information constantly or frequently, and that information may be either streamed in live to the cloud server or collected and sent in chunks to the cloud server via the uplink cellular radio of the respective patient gateway that provides for cellular communication.

The need for reliable cellular communication from a patient gateway to a cloud server is exemplified by an instance in which a patient is alone in their room or walking outdoors. In this instance, the cellular radio of the patient gateway must support both a reliable and quick cellular communications path to facilitate remote monitory of the patient.

As patient gateways are commonly powered by batteries, the conservation of energy by the patient gateways is desired, both to reduce the instances in which the battery must be replaced or recharged and to insure that the patient gateway can remain functional for an extended period of time. The energy consumed during cellular access is only partially dictated based on bytes transmitted from the patient gateway to the cloud server. Another, sometimes more significant factor in power consumption is the frequency of the transmissions and the size of the data that is transmitted. For example in a Long Term Evolution (LTE) network, if discontinuous reception (DRX) timers cause active states to last 10 seconds, the transmission of 1 byte or 100 kilobytes consumes almost the same amount of energy.

### BRIEF SUMMARY

A method, apparatus and computer program product are provided in accordance with an example embodiment in order to provide communication of patient sensor data from patient gateways to a network device and, more particularly, to provide for such communication in an energy efficient manner. For example, the method, apparatus and computer program product of an example embodiment may take advantage of the fact that many patients and, therefore, many patient gateways are nearby to each other. As a result, the method, apparatus and computer program product of an example embodiment may facilitate cooperation between the patient gateways and may provide for the collective communication of patient sensor data to a network device, thereby conserving power consumption by the patient gateways.

In accordance with an example embodiment, a method is provided that includes collecting patient sensor data at a first patient gateway and monitoring for one or more second patient gateways. The method also determines one or more characteristics of each second patient gateway and analyzes the one or more characteristics to determine which of the first patient gateway or the one or more second patient gateways should serve as a cellular uplink gateway to a network device. In an instance in which a respective second patient gateway is determined to serve as the cellular uplink gateway, the method also causes a representation of the patient sensor data to be provided to the respective second patient gateway for subsequent provision to the network device.

The one or more characteristics include, at least in part, battery status, cellular signal strength, status as a cellular uplink gateway, system to which the second patient gateway belongs or a combination thereof. In an example embodiment, the analysis of the one or more characteristics includes firstly analyzing the battery status and secondly, analyzing the cellular signal strength to determine which of the first patient gateway or the one or more second patient gateways should serve as the cellular uplink gateway. Upon determination of the respective second patient gateway to serve as the cellular uplink gateway, the method of this example embodiment further includes causing authentication information to be exchanged with the respective second patient gateway.

In an example embodiment, the method analyzes one or more characteristics by comparing the one or more characteristics to respective threshold or configuration values. The method of this example embodiment also discontinues service of the respective second patient gateway as the cellular uplink gateway based, at least in part, on the comparing. Upon discontinuing service of the respective second patient gateway as the cellular uplink gateway, the method of this example embodiment may also repeat the monitoring for one or more second patient gateways and the subsequent determination of the cellular uplink gateway. The method of an example embodiment also includes maintaining a connection to a network that includes the network device while the respective second patient gateway serves as the cellular uplink gateway.

In another example embodiment, an apparatus is provided that includes at least one processor and at least one memory storing computer program code with the at least one memory and the computer program code configured to, with the processor, cause the apparatus to collect patient sensor data at a first patient gateway and monitor for one or more second patient gateways. The at least one memory and the computer program code are further configured to, with the processor, cause the apparatus to at least determine one or more characteristics of each second patient gateway and analyze the one or more characteristics to determine which of the first patient gateway or the one or more second patient gateways should serve as a cellular uplink gateway to a network device. In an instance in which a respective second patient gateway is determined to serve as the cellular uplink gateway, the at least one memory and the computer program code are further configured to, with the processor, cause a representation of the patient sensor data to be provided to the respective second patient gateway for subsequent provision to the network device.

The one or more characteristics include, at least in part, battery status, cellular signal strength, status as a cellular uplink gateway, system to which the second patient gateway belongs or a combination thereof. The at least one memory and the computer program code are further configured to, with the processor, cause the apparatus of an example embodiment to firstly analyze the battery status and secondly analyze the cellular signal strength to determine which of the first patient gateway or the one or more second patient gateways should serve as the cellular uplink gateway. Upon determination of the respective second patient gateway to serve as the cellular uplink gateway, the at least one memory and the computer program code are further configured to, with the processor, cause the apparatus of this example embodiment to cause authentication information to be exchanged with the respective second patient gateway.

In an example embodiment, the at least one memory and the computer program code are further configured to, with the processor, cause the apparatus of an example embodiment to analyze the one or more characteristics by comparing the one or more characteristics to respective threshold or configuration values. The at least one memory and the computer program code are further configured to, with the processor, cause the apparatus of an example embodiment to discontinue service of the respective second patient gateway as the cellular uplink gateway based, at least in part, on the comparing. Upon discontinuing service of the respective second patient gateway as the cellular uplink gateway, the at least one memory and the computer program code are further configured to, with the processor, cause the apparatus of an example embodiment to also repeat the monitoring for one or more second patient gateways and the subsequent determination of the cellular uplink gateway. The at least one memory and the computer program code are further configured to, with the processor, cause the apparatus of an example embodiment to maintain a connection to a network that includes the network device while the respective second patient gateway serves as the cellular uplink gateway.

In a further example embodiment, a computer program product is provided that includes at least one non-transitory computer-readable storage medium having computer-executable program code instructions stored therein with the computer-executable program code instructions including program code instructions configured to collect patient sensor data at a first patient gateway and monitor for one or more second patient gateways. The computer-executable program code instructions also include program code instructions configured to at least determine one or more characteristics of each second patient gateway and analyze the one or more characteristics to determine which of the first patient gateway or the one or more second patient gateways should serve as a cellular uplink gateway to a network device. In an instance in which a respective second patient gateway is determined to serve as the cellular uplink gateway, the computer-executable program code instructions also include program code instructions configured to cause a representation of the patient sensor data to be provided to the respective second patient gateway for subsequent provision to the network device.

The one or more characteristics include, at least in part, battery status, cellular signal strength, status as a cellular uplink gateway, system to which the second patient gateway belongs or a combination thereof. The computer-executable program code instructions may also include program code instructions configured to firstly analyze the battery status and secondly analyze the cellular signal strength to determine which of the first patient gateway or the one or more second patient gateways should serve as the cellular uplink gateway. Upon determination of the respective second patient gateway to serve as the cellular uplink gateway, the computer-executable program code instructions of this example embodiment may also include program code instructions configured to cause authentication information to be exchanged with the respective second patient gateway.

The computer-executable program code instructions of an example embodiment may also include program code instructions configured to analyze the one or more characteristics by comparing the one or more characteristics to respective threshold or configuration values. The computer-executable program code instructions of an example embodiment may also include program code instructions configured to discontinue service of the respective second patient gateway as the cellular uplink gateway based, at least in part, on the comparing. Upon discontinuing service of the respective second patient gateway as the cellular uplink gateway, the computer-executable program code instructions of an example embodiment may also include program code instructions configured to also repeat the monitoring for one or more second patient gateways and the subsequent determination of the cellular uplink gateway.

In yet another example embodiment, an apparatus is provided that includes means for collecting patient sensor data at a first patient gateway and means for monitoring for one or more second patient gateways. The apparatus also includes means for determining one or more characteristics of each second patient gateway and means for analyzing the one or more characteristics to determine which of the first patient gateway or the one or more second patient gateways should serve as a cellular uplink gateway to a network device. In an instance in which a respective second patient gateway is determined to serve as the cellular uplink gateway, the apparatus also includes means for causing a representation of the patient sensor data to be provided to the respective second patient gateway for subsequent provision to the network device.

### BRIEF DESCRIPTION OF THE DRAWINGS

Having thus described certain example embodiments in general terms, reference will hereinafter be made to the accompanying drawings, which are not necessarily drawn to scale, and wherein:

FIG. 1 is a representation of patient gateways connected to a cloud server over a cellular interface;

FIG. 2 is a block diagram of an apparatus that may be specifically configured in accordance with an example embodiment;

FIG. 3 is a flowchart illustrating operations performed in accordance with an example embodiment of the present invention;

FIG. 4 is a flowchart illustrating operations performed for selecting a patient gateway in accordance with an example embodiment of the present invention;

FIG. 5 is a message sequence for selecting a patient gateway to serve as a cellular uplink gateway in accordance with an example embodiment of the present invention;

FIG. 6 is a representation of patient gateways sending advertisements to each other in accordance with an example embodiment of the present invention;

FIG. 7 is a representation of data flowing through two cellular uplink gateways to a cloud server in accordance with an example embodiment of the present invention;

FIG. 8 is a representation of a reconfiguration of the data flowing through two different cellular uplink gateways to a cloud server in accordance with an example embodiment of the present invention; and

FIG. 9 illustrates the protocol utilized by a first patient gateway and a cellular uplink gateway for the forwarding of patient sensor data in accordance with an example embodiment of the present invention.

### DETAILED DESCRIPTION

Some embodiments will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information,” and similar terms may be used interchangeably to refer to data capable of being transmitted, received and/or stored in accordance with embodiments of the present invention. Thus, use of any such terms should not be taken to limit the spirit and scope of embodiments of the present invention.

Additionally, as used herein, the term ‘circuitry’ refers to (a) hardware-only circuit implementations (e.g., implementations in analog circuitry and/or digital circuitry); (b) combinations of circuits and computer program product(s) comprising software and/or firmware instructions stored on one or more computer readable memories that work together to cause an apparatus to perform one or more functions described herein; and (c) circuits, such as, for example, a microprocessor(s) or a portion of a microprocessor(s), that require software or firmware for operation even if the software or firmware is not physically present. This definition of ‘circuitry’ applies to all uses of this term herein, including in any claims. As a further example, as used herein, the term ‘circuitry’ also includes an implementation comprising one or more processors and/or portion(s) thereof and accompanying software and/or firmware. As another example, the term ‘circuitry’ as used herein also includes, for example, a baseband integrated circuit or applications processor integrated circuit for a mobile phone or a similar integrated circuit in a server, a cellular network device, other network device, and/or other computing device.

As defined herein, a “computer-readable storage medium,” which refers to a non-transitory physical storage medium (e.g., volatile or non-volatile memory device), can be differentiated from a “computer-readable transmission medium,” which refers to an electromagnetic signal.

A method, apparatus and computer program product are provided in accordance with an example embodiment in order to provide patient gateways that are configured to provide an energy efficient mechanism for collectively communicating patient sensor data to a network device, such as through cellular access sharing. By way of example of a conventional implementation, the data volumes from patient gateways are rather small, around 1-10 kB per patient gateway per second. In contrast, cellular networks, such as third generation (3G) and fourth generation (4G) networks, can transmit megabytes of data per second. Hence, the battery consumption of conventional patient gateways is dominated by the frequency of connectivity events rather than the bytes of data transmitted.

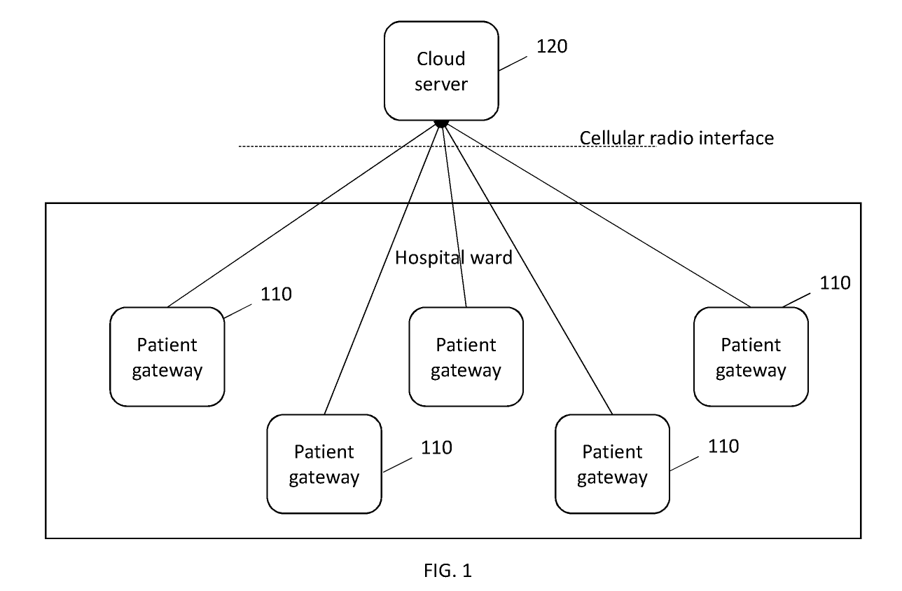

A conventional implementation is shown in FIG. 1 in which a plurality of patient gateways 110, each worn by or otherwise associated with a different patient within a hospital ward, establish a cellular radio connection with a cloud server 120 so as to upload patient data to the cloud server. In the implementation of FIG. 1, the patient gateway runs out of energy more quickly than is desired as a result of the frequent transmission of data over a cellular connection to the cloud server.

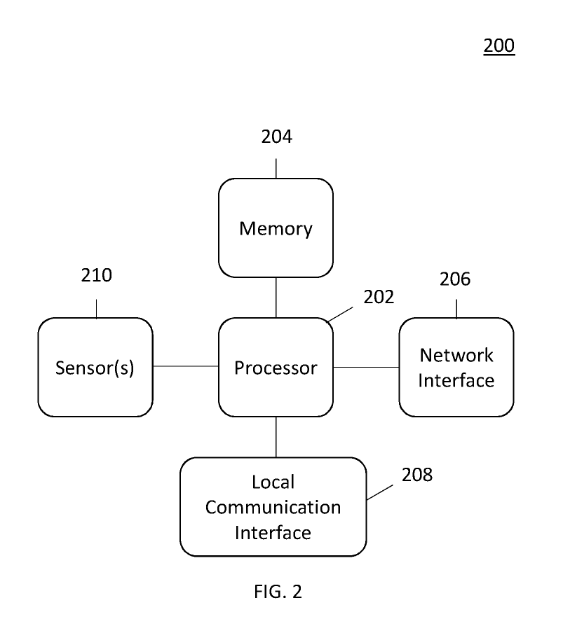

In order to conserve power consumption so as to allow the batteries of the patient gateways to have an extended life between recharging events, a method, apparatus and computer program product are provided in accordance with an example embodiment in order to leverage the proximity of multiple patient gateways such that the patient data collected by one or more patient gateways is transmitted by another one of the patient gateways to a network node, such as a cloud server (or set of cloud servers) for analysis, storage, etc. The apparatus 200 of an example embodiment may be embodied in various manners, but, in an example embodiment, is embodied by a patient gateway. Regardless of the manner in which the apparatus is embodied, the apparatus may include one or more processors 202, one or more memory devices 204, a remote network interface 206 and a local communication interface 208 as shown in FIG. 2. The apparatus may also include or otherwise be in communication with one or more sensors 210 worn by or otherwise associated with a patient so as to provide patient data. As used herein, a patient is an individual who is being monitored for health care purposes, including the monitoring performed prior to and/or following surgery or an illness, during rehabilitation or other treatment or the like. Additionally, the patient data (also known as patient sensor data) may be any of various types of health care data collected by sensors worn by or otherwise monitoring the patient and associated with the patient so as to be utilized in conjunction with the monitoring of the patient for health care purposes.

In some embodiments, the processor 202 (and/or co-processors or any other processing circuitry assisting or otherwise associated with the processor) may be in communication with the memory device 204 via a bus for passing information among components of the apparatus 200. The memory device may be non-transitory and may include, for example, one or more volatile and/or non-volatile memories. In other words, for example, the memory device may be an electronic storage device (e.g., a computer readable storage medium) comprising gates configured to store data (e.g., bits) that may be retrievable by a machine (e.g., a computing device like the processor). The memory device may be configured to store information, data, content, applications, instructions, or the like for enabling the apparatus to carry out various functions in accordance with an example embodiment of the present invention. For example, the memory device could be configured to buffer input data for processing by the processor. Additionally or alternatively, the memory device could be configured to store instructions for execution by the processor.

As described above, the apparatus 200 may be embodied by a patient gateway. However, in some embodiments, the apparatus may be embodied as a chip or chip set. In other words, the apparatus may comprise one or more physical packages (e.g., chips) including materials, components and/or wires on a structural assembly (e.g., a baseboard). The structural assembly may provide physical strength, conservation of size, and/or limitation of electrical interaction for component circuitry included thereon. The apparatus may therefore, in some cases, be configured to implement an embodiment of the present invention on a single chip or as a single “system on a chip.” As such, in some cases, a chip or chipset may constitute means for performing one or more operations for providing the functionalities described herein.

The processor 202 may be embodied in a number of different ways. For example, the processor may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally or alternatively, the processor may include one or more processors configured in tandem via the bus to enable independent execution of instructions, pipelining and/or multithreading.

In an example embodiment, the processor 202 may be configured to execute instructions stored in the memory device 204 or otherwise accessible to the processor. Alternatively or additionally, the processor may be configured to execute hard coded functionality. As such, whether configured by hardware or software methods, or by a combination thereof, the processor may represent an entity (e.g., physically embodied in circuitry) capable of performing operations according to an embodiment of the present invention while configured accordingly. Thus, for example, when the processor is embodied as an ASIC, FPGA or the like, the processor may be specifically configured hardware for conducting the operations described herein. Alternatively, as another example, when the processor is embodied as an executor of software instructions, the instructions may specifically configure the processor to perform the algorithms and/or operations described herein when the instructions are executed. However, in some cases, the processor may be a processor of a specific device (e.g., a patient gateway) configured to employ an embodiment of the present invention by further configuration of the processor by instructions for performing the algorithms and/or operations described herein. The processor may include, among other things, a clock, an arithmetic logic unit (ALU) and logic gates configured to support operation of the processor.

The apparatus 200 also includes both a remote network interface 206 and a local communication interface 208. The remote network interface may be any means such as a device or circuitry embodied in either hardware or a combination of hardware and software that is configured to transmit data to a remote network device, such as a cloud server or a set of servers, such as via a cellular network. Although described herein as a cellular connection, various types of remote network connections may be utilized in other embodiments. Similarly, the local communication network may be any means such as a device or circuitry embodied in either hardware or a combination of hardware and software that is configured to receive and/or transmit data from/to other patient gateways that are located in proximity to the apparatus, such as via any of the various proximity based communication techniques, such as in accordance with Bluetooth™ LE, Bluetooth™, IEEE 802.15.4 or WiFi protocols. The remote network interface and the local communication interface may each include, for example, an antenna (or multiple antennas) and supporting hardware and/or software for enabling communications with a wireless communication network. Additionally or alternatively, the remote network interface and the local communication interface may each include the circuitry for interacting with the antenna(s) to cause transmission of signals via the antenna(s) or to handle receipt of signals received via the antenna(s).

The apparatus 200 of the example embodiment may include or otherwise be associated with one or more sensors 210. The sensors may be configured to measure, monitor or otherwise collect various different types of patient data. For example the sensors may include a temperature sensor, a blood pressure sensor, an oxygen sensor or the like. The apparatus may include other types of sensors in other embodiments.

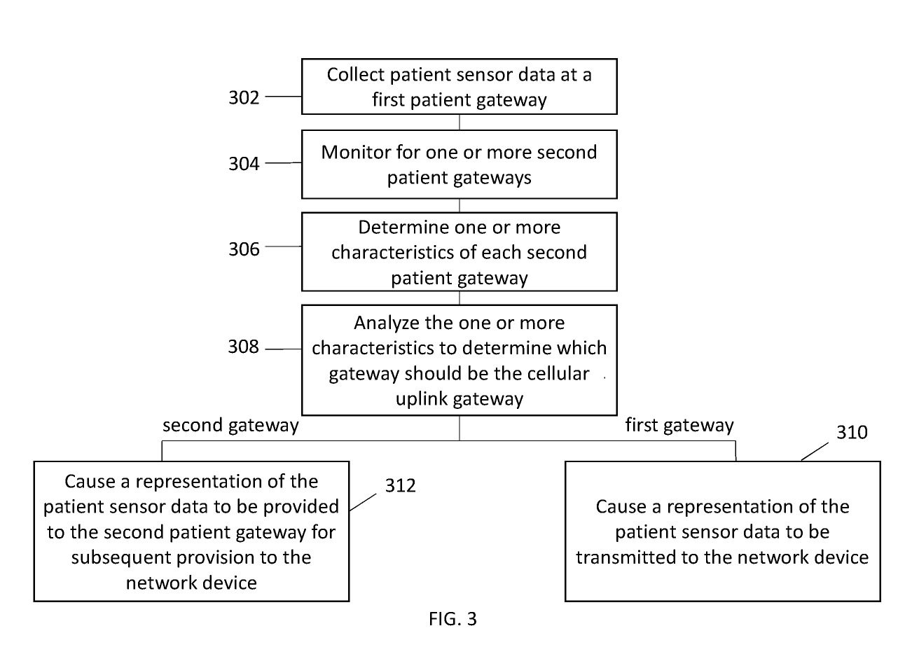

Referring now to FIG. 3, the operations performed, such as by the apparatus 200 of FIG. 2, in order to provide communication of patient sensor data from patient gateways to a network device in an energy efficient manner. As shown in block 302, the apparatus 200 embodied by a first patient gateway includes means, such as the processor 202 or the like, for collecting patient sensor data from the sensors 210. The patient sensor data may be stored, at least temporarily, by the memory 204. Although shown as a single block, the patient sensor data may be collected in an ongoing or a repeated manner, such as in accordance with a predefined schedule.

As shown in FIG. 3, in block 304, the apparatus 200 embodied by the first patient gateway includes means, such as the processor 202, the local communication interface 208 or the like, for monitoring for one or more second patient gateway(s). In this regard, the second patient gateways that are in proximity to the first patient gateway, such as by being within communication range of the local communication interface 208 and/or being capable of communicating with the first patient gateway with signals having a signal strength or a signal to noise ratio that satisfy a predefined threshold, may be identified by the processor of the first patient gateway as candidates to serve as a cellular uplink gateway.

As shown in block 306, the apparatus 200 embodied by the first patient gateway also includes means, such as the processor 202, the local communication interface 208 or the like, for determining one or more characteristics of each second patient gateway that was identified by the monitoring of block 304. In this regard, the processor of an example embodiment is configured to query each second patient gateway that is identified by the monitoring as to its battery status, its cellular signal strength, whether the second patient gateway is currently serving a cellular uplink gateway, the system to which the second patient gateway belongs and/or if the second patient gateway is willing to serve as a cellular uplink gateway for the first patient gateway. The apparatus further includes means, such as the processor or the like, for analyzing the characteristic(s) of the second patient gateway(s) to determine which of the first patient gateway or the one or more second patient gateways should serve as a cellular uplink gateway to a network device. See block 308. In an example embodiment, the apparatus, such as the processor, is configured to analyze the one or more characteristics by comparing the one or more characteristics to respective threshold or configuration values. For example, the characteristics that are expressed in terms of a numerical value, such as battery status and cellular signal strength, may be compared to a predefined threshold. As another example, the characteristics that are expressed in non-numerical terms, such as whether the second patient gateway is currently serving a cellular uplink gateway, the system to which the second patient gateway belongs and whether the second patient gateway is willing to serve as a cellular uplink gateway for the first patient gateway may be compared to a predefined configuration value. With respect to the characteristic relating to the system to which the second patient gateway belongs, the characteristic may indicate that the second patient gateway belongs to the same system as the first patient gateway (such as by belonging to the same hospital or administrative domain), or to a different system than the first patient gateway.

As described below, the determination as to which of the first patient gateway or the one or more second patient gateways should serve as a cellular uplink gateway to a network device may be based on an analysis of the various combinations of the characteristics. For example, a second patient gateway that is currently serving as a cellular uplink gateway, belongs to the same system as the first patient gateway and is also willing to serve as a cellular uplink gateway for the first patient gateway may be selected as the cellular uplink gateway for the first patient gateway. Alternatively, the gateway having the greatest cellular signal strength, the most remaining battery power or some combination thereof may be selected as the cellular uplink gateway for the first patient gateway.

In an instance in which the first patient gateway is determined to be the gateway to serve as the cellular uplink gateway, the apparatus 200 embodied by the first patient gateway may also include means, such as the processor 202, the remote network interface 206 or the like, for causing a representation of the patient sensor data collected by the first patient gateway, such as the actual patient sensor data, a summary of the patient sensor data, an encrypted representation of the patient sensor data or the like, to be transmitted to the network device, such as for analysis, storage, etc. See block 310. In this instance, the first patient gateway may also serve as the cellular uplink gateway for one or more of the second patient gateways such that the first patient gateway may also be configured to receive patient sensor data from one or more second patient gateways and may then also forward the patient sensor data from the one or more second patient gateways to the network device. Alternatively, in an instance in which a respective second patient gateway is determined to be the gateway to serve as the cellular uplink gateway, the apparatus 200 embodied by the first patient gateway may also include means, such as the processor 202, the local communication interface 208 or the like, for causing a representation of the patient sensor data collected by the first patient gateway, such as the actual patient sensor data, a summary of the patient sensor data, an encrypted representation of the patient sensor data or the like, to be provided to the respective second patient gateway for subsequent provision by the respective second patient gateway to the network device. See block 312. By utilizing the lower powered local communication interface to provide the patient sensor data to the second patient gateway instead of the using the remote network interface to provide the patient sensor data to the network device, the first patient gateway consumes less power, thereby increasing the lifetime of its battery. In this instance, the respective second patient gateway serves as the cellular uplink gateway for itself and the first patient gateway and perhaps one or more other second patient gateways. Thus, the transmissions to the network device may be consolidated, such that the cellular uplink gateway provides more patient sensor data to the network device than any one patient gateway, but may the number of transmissions may not increase, at least not appreciably, relative to those of any one patient gateway such that the power consumed by the patient gateways on a collective basis is reduced relative to a conventional implementation in which each patient gateway transmits its patient sensor data to the network device. In an example embodiment in which a second patient gateway serves as the cellular uplink gateway, the first patient gateway, such as the processor, the network interface 206 or the like, may be configured to maintain a connection with the network that includes the network device, albeit a connection that consumes significantly less power than that consumed while serving as the cellular uplink gateway.

Although described herein as a cellular uplink gateway, the uplink gateway may utilize other types of remote network connections, different than a cellular connection, in other embodiments. As such, reference herein to a cellular uplink gateway is provided by way of an example of an uplink gateway serving two or more patient gateways utilizing a remote, e.g., non-proximity based, connection, such as a cellular or other type of remote connection.

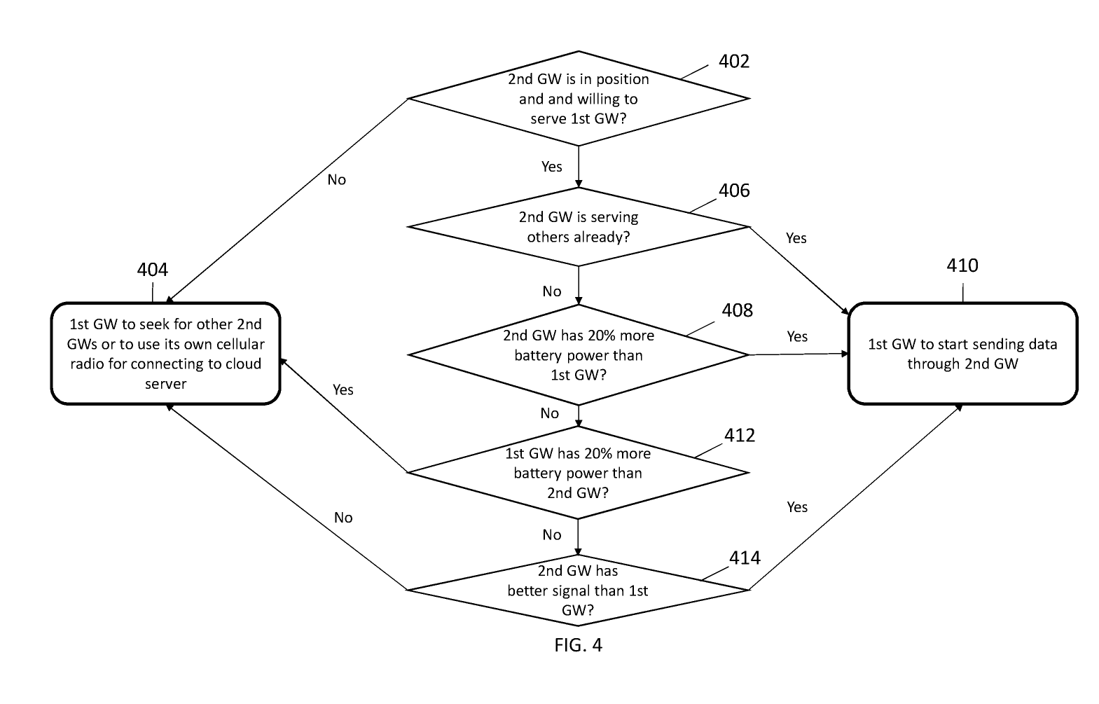

By way of one example of the determination of the patient gateway (GW) that will serve as the cellular uplink gateway, reference is made to FIG. 4. Upon identifying a second patient gateway as being proximate the first patient gateway, the first patient gateway queries the second patient gateway to determine if the second patient gateway is in position (e.g., is able to establish a cellular uplink connection with the cellular uplink gateway and has sufficient battery, memory and processing resources to serve as a cellular uplink gateway) and is willing to serve the requesting first patient gateway as shown in block 402. If the second patient gateway is not in position or is unwilling to serve the first patient gateway, the first patient gateway responds by seeking another second patient gateway for service as a cellular uplink gateway or using its own cellular radio for connecting to the network device as shown in block 404. In the instance when the second patient gateway is willing to serve the first patient gateway, the first patient gateway of this embodiment queries the respective second patient gateway as to whether the second patient gateway is already serving other patient gateways as the cellular uplink gateway. See block 406. In the instance in which the second patient gateway is already serving as a cellular uplink gateway, the first patient gateway determines that the second patient gateway will also serve as its cellular uplink gateway and starts to send a representation of the patient sensor data through the second patient gateway as shown in block 410.

In the instance when the second patient gateway is not already serving as the cellular uplink gateway, the first patient gateway queries the second patient gateway and then firstly analyzes the battery status and secondly analyzes the cellular signal strength to determine which patient gateway should serve as the uplink cellular gateway. In the illustrated embodiment, the first gateway is configured to determine whether the second patient gateway has more battery power, such as by a predefined amount, e.g., 20%, than the first patient gateway as shown in block 408. If the second patient gateway does have sufficiently more battery power, the first patient gateway can determine that the second patient gateway will also serve as its cellular uplink gateway and start sending a representation of the patient sensor data through the second patient gateway. See block 410. In the instance in which the second patient gateway does not have sufficiently more battery power, however, the first patient gateway of this example embodiment determines whether it has more battery power, such as by a predefined amount, e.g., 20%, than the second patient gateway as shown in block 412. If it is determined that the first patient gateway does have sufficiently more battery power, the first patient gateway is configured to seek another second patient gateway for service as a cellular uplink gateway or to use its own cellular radio for connecting to the network device as shown in block 404.

If the first patient gateway does not have sufficiently more batter power than the second patient gateway, the first patient gateway of this embodiment is configured to query the second patient gateway to determine whether the second patient gateway has a better cellular signal, such as by having a greater cellular signal strength and/or a greater signal to noise ratio, than the first patient gateway as shown in block 414. If the second patient gateway does have a better cellular signal, the first patient gateway begins sending a representation of the patient sensor data through the second patient gateway as shown in block 410. However, in the instance in which the second patient gateway does not have a better cellular signal, the first gateway may be again configured to seek another second patient gateway for service as a cellular uplink gateway or to use its own cellular radio for connecting to the network device as shown in block 404.

In an example embodiment, the patient gateways may be configured to authenticate each other using any of a variety of authentication techniques, such as cloud-based authentication or the exchange of pre-configured shared keys or secrets. For example, when patient gateways are deployed on a hospital ward, the patient gateways could be provided with credentials that the patient credentials need to communicate with each other for cellular uplink sharing purposes. In an example embodiment, these credentials would only be used for power optimization and access sharing, not accessing patient data on patient gateways such that the confidentiality of the patient data is maintained. In this regard, in an embodiment in which a second patient gateway is selected to serve as the cellular uplink gateway, the first patient gateway may forward patient data to the second patient gateway for forwarding to a network device, such as a cloud server. The patient data that is forwarded from the first patient gateway to the second patient gateway may be encrypted or otherwise secured. Although the network device may be configured to access the patient data upon its receipt, such as by decrypting the patient data, the second patient gateway may be unable to decrypt or otherwise access the patient data provided by the first patient gateway, thereby protecting the confidentiality of the patient data.

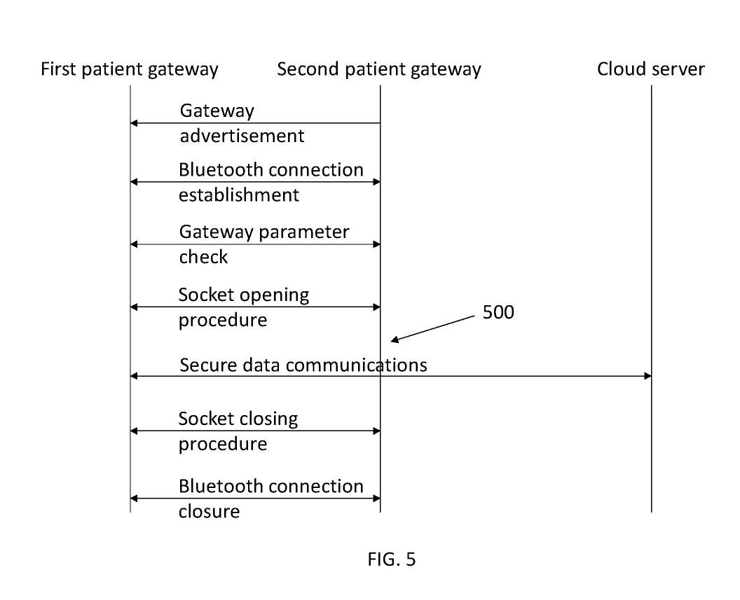

Although the patient gateway may exchange various types of messages to determine which patient gateway is to serve as the cellular uplink gateway, FIG. 5 illustrates an example high-level message exchange between first and second patient gateways. In this embodiment, both patient gateways initially have a cellular connection. However, in an instance in which the second patient gateway does not initially have a cellular connection, the second patient gateway will open a cellular connection at latest at point 500.

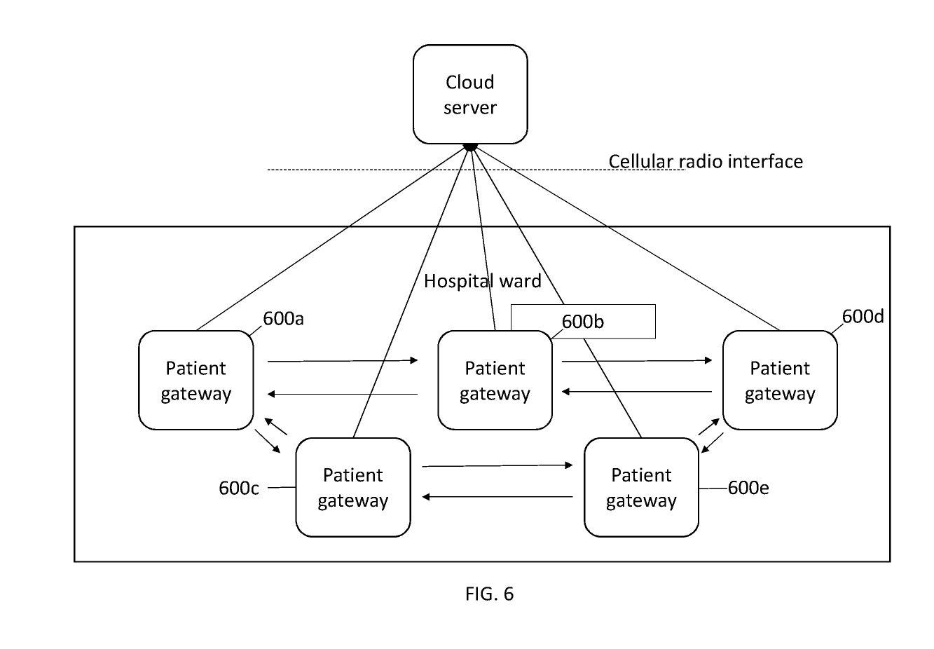

In an example embodiment, all patient gateways may issue gateway advertisements. However, not every patient gateway hears or receives the gateway advertisement issued by every other patient gateway. The proximity based connection, such as a Bluetooth™ connection, can be established between the patient gateways that hear one another, as indicated by receipt of the gateway advertisements. As shown in FIG. 6, for example, the Bluetooth connections that are established are shown with directional lines between respective patient gateway pairs. Thus, patient gateway 600a hears the gateway advertisements from patient gateway 600b and 600c and establishes Bluetooth™ connections therewith. However, patient gateway 600a does not hear the gateway advertisements from patient gateway 600d and 600e and, as a result, does not establish Bluetooth™ connections therewith. The manner in which a patient gateway connects to other patient gateways can vary. For example, patient gateways may connect to some, but not all, patient gateways from which the gateway advertisements were received, such as by connecting to the first gateway from which an advertisement was heard. In another example, the first patient gateway could connect to more than one or every second patient gateway from which gateway advertisements were heard, and then determine the most suitable patient gateway to act as the cellular uplink gateway.

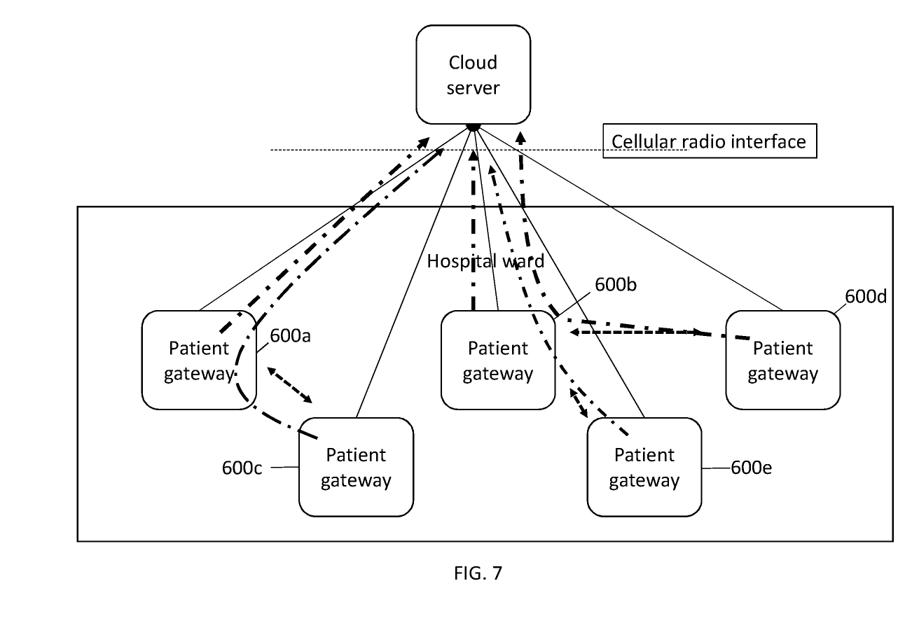

As shown in FIG. 5, after establishing the Bluetooth™ connections, the parameters, e.g., characteristics, of the patient gateways may be checked. The first patient gateway may then select the second patient gateway to serve as its cellular uplink gateway. Thus, in the illustrated embodiment, a socket may be opened between the first and second patient gateways to facilitate the transfer of patient data from the first patient gateway to the second patient gateway and, in turn, to the network device, e.g., cloud server. As shown in FIG. 7, for example, patient gateway 600a may be selected to serve as the cellular uplink gateway for both itself and patient gateway 600c. Additionally, patient gateway 600b may be selected to serve as the cellular uplink gateway for itself, patient gateway 600d and patient gateway 600e. Following the completion of the transfer of patient data on behalf of the first patient gateway, the socket may be closed and the Bluetooth™ connection may be similarly closed.

In an example embodiment, the patient gateways will keep their own cellular connection open and active all the time, in order to provide quick fallback and better accessibility from the network device when actuation or data reading initiated from the network needs to be performed as quickly as possible. The maintenance of an open and active cellular connection also allows for fast response times if another patient gateway that is selected to serve as the cellular uplink gateway (or the local communication link therewith) ceases to work. Idle cellular connection consumes little energy.

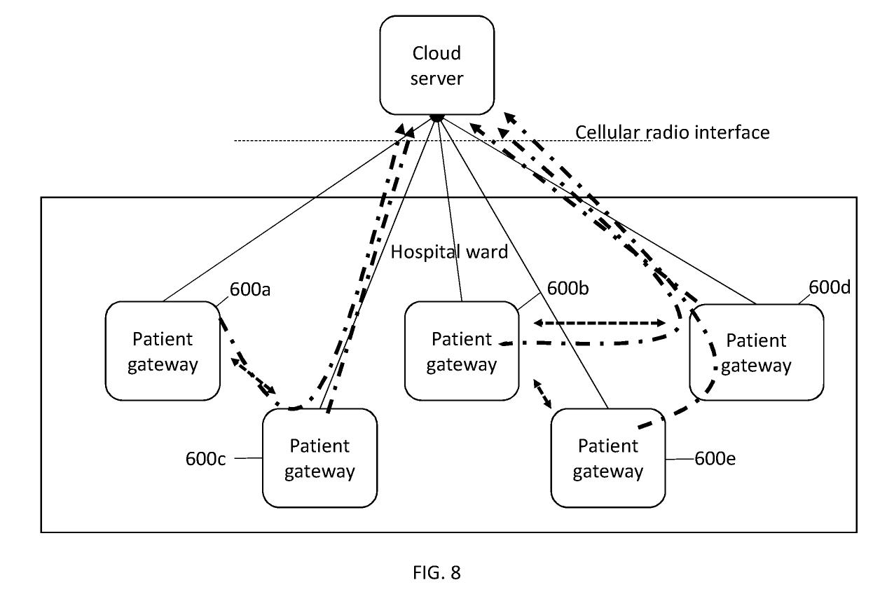

Once a cellular uplink gateway has been selected and is functional in relation to forwarding patient sensor data to the network device, the first gateway device or the gateway devices that have been selected as the cellular uplink gateways may repeatedly determine if the current selection for the cellular uplink gateway continues to be appropriate or if the current selection should be discontinued and another patient gateway should serve as the cellular uplink gateway, such as by repeating the process of FIG. 3 including an analysis of various characteristics of the patient gateways as described above. In the embodiment of FIG. 7, it may be determined that patient gateways 600a and 600b no longer have sufficient resources to continue to serve as the cellular uplink gateways, such as by battery levels that fall below a 50% battery level, or that are 20% less than the battery level of other patient gateways. This causes rediscovery of patient gateways and another selection of a patient gateway to serve as the cellular uplink gateway. As shown in FIG. 8, for example, patient gateways 600c and 600d may now be selected to serve as the cellular uplink gateways.

In an example embodiment, the patient gateways may be configured to utilize a protocol for selecting a cellular uplink gateway, or the patient gateways could form a mesh network, such as a Bluetooth™ LE mesh network, to help assist in the selection of the cellular uplink gateway. While various protocols may be utilized to exchange data between the patient gateways, the protocol used for transmitting data between patient gateways in an example embodiment can be Bluetooth™ BD/Enhanced Data Rate (EDR), e.g. using a serial profile, Bluetooth™ LE using Generic Attribute Profile (GATT), or transmitting data directly on top of Logical Link Control and Adaptation (L2CAP) or a set of different protocols can be used with Bluetooth™ or Bluetooth™ LE, or if another local connectivity radio is used such as 802.11 WiFi or 802.15.4. If IPv6 over Bluetooth (LE) is used, each gateway could send the battery and cellular signal status in multicast messages, which would assist other patient gateways to decide if they should forward patient sensor data through another gateway.

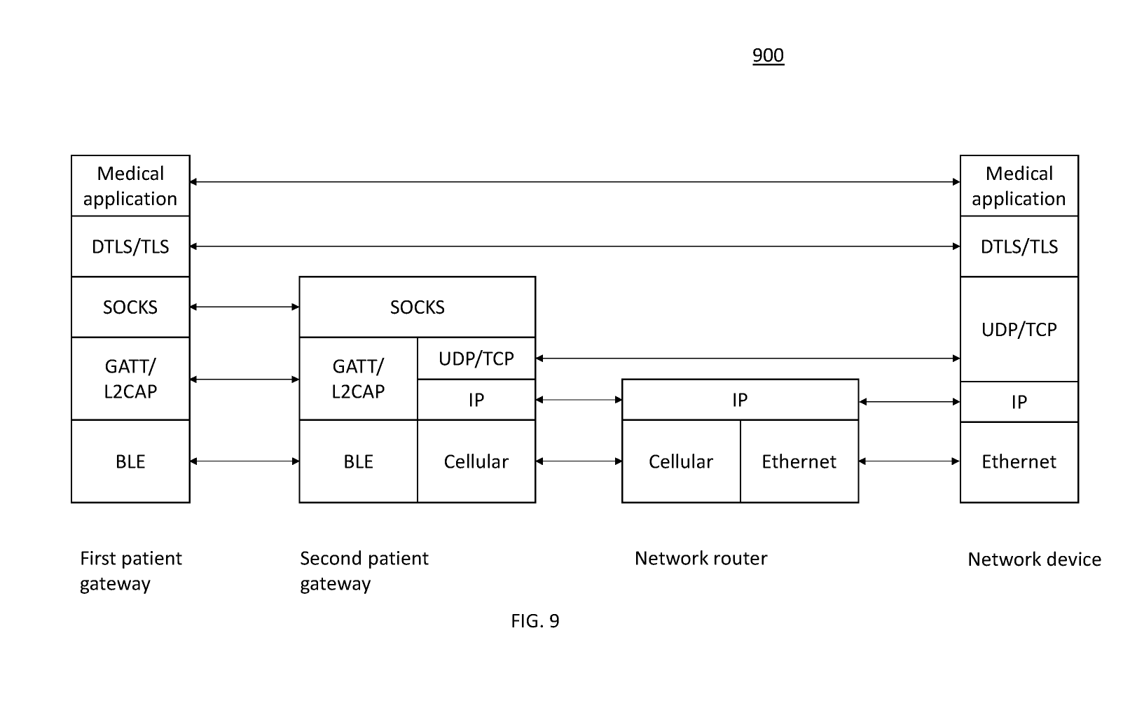

By way of example, but not of limitation, of the protocol, the patient gateway that wishes to use another patient gateway as the cellular uplink gateway could utilize a Socket Secure (SOCKS)-based approach, in which the cellular uplink gateway creates a Transmission Control Protocol (TCP)/User Datagram Protocol (UDP) socket to the network device, and the first patient gateway passes application layer data to cellular uplink gateway for putting into the TCP/UDP socket. This allows the implementation of a transport layer security (TLS) or a datagram transport layer security (DTLS) session between the first patient gateway and network device, and hence does not allow second patient gateway serving as the cellular uplink server to see into data. This stack is illustrated in FIG. 9 using Bluetooth LE (BLE) as example radio protocol between the patient gateways, a cellular connection between the second patient gateway and a network router, and an Ethernet connection between the network router and a network device, such as a cloud server. Other protocols than SOCKS can be used as well, but SOCKS is shown in this example embodiment as it is established protocol for proxying purposes.

Additionally or alternatively, the second patient gateway that serves as the cellular uplink gateway may advise to first patient gateway when the the first patient gateway should forward patient sensor data through the second patient gateway, such as to improve or optimize the power efficiency and/or the memory consumption in order to be optimal both in power consumption and for second gateway memory consumption-wise so that second gateway would not have to buffer much data.

As described above, the method, apparatus and computer program product of an example embodiment provide communication of patient sensor data from patient gateways to a network device in an energy efficient manner by selecting one or more of the patient gateways to serve as a cellular uplink gateway in order to forward patient sensor data for a plurality of the patient gateways to a network device. Thus, the patient gateways may conserve power in order to extend their battery lifetime.

It will be understood that each block of the flowcharts, and combinations of blocks in the flowcharts, may be implemented by various means, such as hardware, firmware, processor, circuitry, and/or other devices associated with execution of software including one or more computer program instructions. For example, one or more of the procedures described above may be embodied by computer program instructions. In this regard, the computer program instructions which embody the procedures described above may be stored by the memory device 204 of an apparatus employing an embodiment of the present invention and executed by the processor 202 of the apparatus. As will be appreciated, any such computer program instructions may be loaded onto a computer or other programmable apparatus (e.g., hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart blocks.

Accordingly, blocks of the flowcharts support combinations of means for performing the specified functions and combinations of operations for performing the specified functions for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

In some embodiments, certain ones of the operations above may be modified or further amplified. Furthermore, in some embodiments, additional optional operations may be included. Modifications, additions, or amplifications to the operations above may be performed in any order and in any combination.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1-20. (canceled)

21. A method comprising:
collecting patient sensor data at a first patient gateway;
monitoring for one or more second patient gateways;
determining one or more characteristics of each second patient gateway;
analyzing the one or more characteristics to determine which of the first patient gateway or the one or more second patient gateways should serve as a cellular uplink gateway to a network device; and
in an instance in which a respective second patient gateway is determined to serve as the cellular uplink gateway, causing a representation of the patient sensor data to be provided to the respective second patient gateway for subsequent provision to the network device.

22. A method of claim 21,
wherein the one or more characteristics include, at least in part, battery status, cellular signal strength, status as a cellular uplink gateway, system to which the second patient gateway belongs or a combination thereof.

23. A method of claim 22,
wherein analyzing the one or more characteristics comprises firstly analyzing the battery status and secondly analyzing the cellular signal strength to determine which of the first patient gateway or the one or more second patient gateways should serve as the cellular uplink gateway.

24. A method of claim 21,
wherein upon determination of the respective second patient gateway to serve as the cellular uplink gateway, the method further comprises causing authentication information to be exchanged with the respective second patient gateway.

25. A method of claim 21,
wherein analyzing the one or more characteristics comprises comparing the one or more characteristics to respective threshold or configuration values, and wherein the method further comprises discontinuing service of the respective second patient gateway as the cellular uplink gateway based, at least in part, on the comparing.

26. A method of claim 25,
wherein upon discontinuing service of the respective second patient gateway as the cellular uplink gateway, the method further comprises repeating the monitoring for one or more second patient gateways and the subsequent determination of the cellular uplink gateway.

27. A method of claim 21, further comprising maintaining a connection to a network that includes the network device while the respective second patient gateway serves as the cellular uplink gateway.

28. An apparatus comprising at least one processor and at least one memory storing computer program code, the at least one memory and the computer program code configured to, with the processor, cause the apparatus to at least:
collect patient sensor data at a first patient gateway;
monitor for one or more second patient gateways;
determine one or more characteristics of each second patient gateway;
analyze the one or more characteristics to determine which of the first patient gateway or the one or more second patient gateways should serve as a cellular uplink gateway to a network device; and
in an instance in which a respective patient gateway is determined to serve as the cellular uplink gateway, cause a representation of the patient sensor data to be provided to the respective second patient gateway for subsequent provision to the network device.

29. An apparatus according to claim 28
wherein the one or more characteristics include, at least in part, battery status, cellular signal strength, status as a cellular uplink gateway, system to which the second patient gateway belongs or a combination thereof.

30. An apparatus according to claim 29 wherein to analyze the one or more characteristics, the at least one memory and the computer program code are further configured to, with the processor, cause the apparatus to:
analyze the battery status, and analyze the cellular signal strength to determine which of the first patient gateway or the one or more second patient gateways should serve as the cellular uplink gateway.

31. An apparatus according to claim 28 wherein to to determine the respective second patient gateway to serve as the cellular uplink gateway, the at least one memory and the computer program code are further configured to, with the processor, cause the apparatus to:
cause authentication information to be exchanged with the respective second patient gateway.

32. An apparatus according to claim 28 wherein to analyze the one or more characteristics, the at least one memory and the computer program code are further configured to, with the processor, cause the apparatus to:
compare the one or more characteristics to respective threshold or configuration values, and
discontinue service of the respective second patient gateway as the cellular uplink gateway based, at least in part, on the result of the comparison.

33. An apparatus according to claim 32 wherein to discontinue service of the respective second patient gateway as the cellular uplink gateway, the at least one memory and the computer program code are further configured to, with the processor, cause the apparatus to:
repeat the monitoring for one or more second patient gateways and the subsequent determination of the cellular uplink gateway.

34. An apparatus according to claim 28 wherein the at least one memory and the computer program code are further configured to, with the processor, cause the apparatus to: maintain a connection to a network that includes the network device while the respective second patient gateway serves as the cellular uplink gateway.

35. A computer program product comprising at least one non-transitory computer-readable storage medium having computer-executable program code instructions stored therein, the computer-executable program code instructions comprising program code instructions configured to:
collect patient sensor data at a first patient gateway;
monitor for one or more second patient gateways;
determine one or more characteristics of each second patient gateway;
analyze the one or more characteristics to determine which of the first patient gateway or the one or more second patient gateways should serve as a cellular uplink gateway to a network device; and
in an instance in which a respective second patient gateway is determined to serve as the cellular uplink gateway, cause a representation of the patient sensor data to be provided to the respective second patient gateway for subsequent provision to the network device.

36. A computer program product according to claim 35 wherein the one or more characteristics include, at least in part, battery status, cellular signal strength, status as a cellular uplink gateway, system to which the second patient gateway belongs or a combination thereof.

37. A computer program product according to claim 36 wherein the computer-executable program code instructions further comprise program code instructions configured to analyze the one or more characteristics by firstly analyzing the battery status and secondly analyzing the cellular signal strength to determine which of the first patient gateway or the one or more second patient gateways should serve as the cellular uplink gateway.

38. A computer program product according to claim 35 wherein to determine the respective second patient gateway to serve as the cellular uplink gateway, the computer-executable program code instructions further comprise program code instructions configured to further cause authentication information to be exchanged with the respective second patient gateway.

39. A computer program product according to claim 35 wherein to analyze the one or more characteristics, the computer-executable program code instructions further comprise program code instructions configured to compare the one or more characteristics to respective threshold or configuration values, and wherein the program code instructions configured to discontinue service of the respective second patient gateway as the cellular uplink gateway based, at least in part, on the comparing.

40. A computer program product according to claim 35 wherein to discontinue service of the respective second patient gateway as the cellular uplink gateway, the computer-executable program code instructions further comprise program code instructions configured to repeat the monitoring for one or more second patient gateways and the subsequent determination of the cellular uplink gateway.
